(19) United States
(12) Patent Application Publication
(10) Pub. No.: US 2017/0084191 A1
(43) Pub. Date: Mar. 23, 2017

(54) A Method for Controlling an Individualized Video Data Output on a Display Device and System

(71) Applicant: MassineBoecker GmbH, Berlin (DE)

(72) Inventor: Dirk Boecker, Berlin (DE)

(73) Assignee: MassineBoecker GmbH, Berlin (DE)

(21) Appl. No.: 15/126,014

(22) PCT Filed: Mar. 16, 2015
(86) PCT No.: PCT/EP2015/055459

§ 371 Date: Sep. 14, 2016

(30) Foreign Application Priority Data
Mar. 14, 2014 (EP) PCT/EP2014/055151

(51) Int. Cl. G09B 19/00; A61B 5/16; G09B 5/02

(52) U.S. Cl. CPC G09B 19/00; G09B 5/02; G09B 19/0092; A61B 5/165

(57) ABSTRACT

The disclosure relates to a method and a system for controlling an individualized video data output on a display device. The System further comprises an input device configured to receive present individual performance data and a control device functionally connected to the input device and the display device. The method comprises providing individualized video data output information in the control device, the individualized video data output information comprising a group of different video data output presentations and assigning a group of individual sets of performance parameters to the group of the different video data output presentations, thereby, for each of the individual sets of performance parameters defining an assigned video data output presentation selected from the group of different video data output presentations, receiving present individual performance data in the control device via the input device, the present individual performance data being indicative of a present performance of an individual, determining present individual performance parameters by processing the present personal performance data by the control device, determining a present video data output presentation from the group of different video data output presentations by comparing the present individual performance parameters to the individual sets of performance parameters, and generating video control signals in the control unit, the video control signals being configured to cause output of the present video data output representation on the display device, and presenting the present video data output representation on the display device.

The invention relates to a method for controlling an individualized video data output on a display device and a system and integrating that method in a system for self management improvement.

### BACKGROUND

In recent decades, the fields of healthcare and patient treatment have seen enormous technological progress and success. Conversely, advances in physician/patient communication and support of patient self-management have evolved at a far slower pace. Effective directives to patients and assistance to reach a high level of self-management support are rare. Despite the fact that there is a plethora of systems (APPS, software, devices) available, which address all sorts of aspects necessary for self-management; the offered systems are point solutions, not integrated, and not providing higher-level advice or guidance.

Health Plans and Disease Management Organizations continue to use legacy systems that lack the sophistication of current mobile Health technology opportunities. They generally reach out to their targeted member populations with broad-scale information and interventions via costly call centers staffed with nurses and other clinicians or experts. Personalized and high frequency outreach and individually tuned interventions are missing due to related high cost of conventional methods and the lack of tools to effectively measure and integrate personality, preferences, and abilities of patients.

All in all, today's Population Management efforts from large or small players are inefficient and ineffective and patients are mostly left alone with a set of non integrated and ineffective self-management tools. Motivation is too low and related therapy adherence and overall compliance remains far below desired levels.

People with chronic diseases often suffer from situational depression and need assistance to reverse their down-spiraling life situation. Once this major hurdle of situational depression and/or lethargy has been overcome people open up to an optimization of their situation. Once started it is important to keep the momentum of the improvement steady and sort of provide a ‘hand-holding’ to the individual's progress without being intrusive into anyone's private life.

People suffering chronic conditions require life-long support. The key part is that this support has to adapt to their actual situation and adjusts dynamically to progress or decline in many areas, like health, education, motivation, social environment etc. A dynamic adaptation of support and/or intervention is mandatory to ensure continued interest and acceptance for any long-term support.

Human beings require ‘meaning’ and mostly focus on the big picture. However, people with chronic diseases often have a shorter horizon of their life outlook and thus their behavior can be positively triggered with indicators reflecting short-term accomplishments. Interventions and support systems can be designed in this regard that they offer a process of small steps to eventually accomplish bigger goals (‘baby-step’ concept of many behaviorism theorists) [Habit Design: Resurrecting Behaviorism for Fun and Profit—http://oaklandfuturist.com/habit-design-resurrecting-behaviorism-for-fun-and-profit/#sthash.8VPtTsh7.dpbs [October 2013]]. Venturing large goals in one or only a few steps will compromise overall motivation and often provide reasons for giving up in view of the unattainable. Repeated focusing on smaller steps initiated by comfortable triggers building on the person's actual abilities is the better approach. People only can do what they are capable of doing and the system proposed may be considered a kind of step-wise reward system, which is sustainable and can help people to ‘get moving’ and continue over a long period of time.

Following the theory of psychological types, which was introduced in the 1920s by Carl G. Jung, and the Meyers-Briggs system from the 1940s [http://www.myersbriggs.org], decision making of individuals is strongly influenced by their actual character. It is important to realize that suffering from chronic diseases influences and can shift this genuine set-up of personality and character traits in a certain psychological direction as we have shown in several studies. Certain brain functions important for decision making can be stimulated in different ways. There is a so-called Neuro-IPS method, which is known as such and which integrates Jung's and Meyers-Briggs insights and builds on J. Kuhl's personality assessment system.

### SUMMARY

It is the object of the invention to provide improved technologies for self-care management.

According to the invention a method for controlling an individualized video data output on a display device of a system according to claim 1 is provided. Also, a system for controlling an individualized video data output on a display device according to claim 7 is provided. Further, a computer program product according to claim 8 is provided.

According to one aspect, a method for controlling an individualized video data output on a display device of a system is provided. The system further comprises an input device configured to receive present performance data and a control device functionally connected to the input device and the display device. The input device may be used for receiving user input and/or input from another device connectable to the input device, such as a measuring device. The control device may comprise a data processing unit and a data storage device. The input device may receive data indicating information about a person's condition from an external source, and such data may be submitted for computation and/or integration to the control device.

In the course of the method individualized or personalized video data output information is provided in the control device. The individualized video data output information comprises a group of different video data output representations, e.g. an icon or a picture. Each of the video data output representations is representing a different video output on a display.

Further, the individualized video data output information is assigning a group of individualized sets of performance parameters to the group of the different video data output representation representations. Thereby, an assigned video data output representation selected from the group of different video data output representations is determined for each of the individualized sets of performance parameters. The assigned video data output representation may be determined to represent an integrated status of currently selected and individualized sets of performance characteristics.

Present individualized or individual performance data are received in the control device via the input device. The present individualized or individual performance data are indicative of a present performance of an individual. The performance of the individual, for example, may refer to physical and/psychological conditions (characteristics) of the individual. The performance or performance level of the individual is determined based on a selection of characteristics selected from a group of characteristics. The present performance data represent present values of the characteristics selected for performance level determination.

Following, present individualized performance parameters are determined by processing the present personalized performance data in the control device in real time or with a time delay. The personalized performance parameters are provided for comparing it to the sets of performance parameters, each of which is assigned to a video data output representation. A present video data output representation is determined from the group of different video data output representations by comparing the present personalized performance parameters derived from the present individual performance data to the sets of performance parameters assigned to the video data output representations. The sets of performance parameters assigned to the group of different video data output representations may be pre-stored in the data storage device of the control device.

The present video data output representation is a visual representation of the present performance or performance level determined based on the present selection of characteristics. Such selection may be changed.

In an alternative, an overall performance of the individual may be determined based on an individualized set of physiological, physical and/or psychological conditions (characteristics) for which present data are collected. Present performance data for a selection of characteristics, which selection may vary and be changed over time, may be integrated and processed in the control device to determine an actual overall performance of the individual. Present performance data may be received in the control device via the input device. An overall personalized performance indicated by performance parameters derived from the present performance data may be processed in the control device and may be compared to the sets of parameters assigned to the different video data output representations available for determining a present one from the group of different video data output representations to be presented on the display device.

Video control signals are generated in the control unit. The video control signals generation may be done in response to the determination of the present video data output representation. The video control signals are configured to cause output of the present video data output representation on the display device. Following, the present video data output representation is outputted on the display device.

According to another aspect, a system for controlling an individualized video data output on a display device is provided. The system comprising a display device, an input device configured to receive present individualized performance data, and a control device functionally connected to the input device and the display device. The system is configured to implement a method for controlling an individualized video data output on a display device of the system.

According to a further aspect, a computer program product stored on a storage medium and configured to perform the method for controlling an individualized video data output on a display device during operation on a data processing system is provided.

The method and system provide tools for self-management of an individual, e.g. a patient. The actual performance of the individual is indicated by present values of the characteristics selected for determining the performance or performance level. Depending on the actual performance of the individual an individualized response is provided to the individual by means of individualized video data representation on the individual's display device. For doing so present performance data received by the input device for a selected set of performance characteristics is evaluated. The present performance parameter derived from the present performance data may be compared in the control device to the respective performance criteria and/or threshold values for those performance criteria.

The system may be implemented in a client-server system, the control device being provided in a central server device. The central server device may be configured to receive present individualized performance data for a plurality of individuals. The central server device may submit the video control signals generated to one or more display devices for outputting the present video data output representation. A self-care management system may be implemented by using the client server architecture. The individualized or individual performance data may be captured by any device configured for user data input like tablet computer, watch, handheld display device, laptop computer, diagnostic devices, TV-box, measuring wearable device, and personal computer. Alternatively, individual performance data may be received from another source like a data storage unit and/or a computer system of a third party, which may submit performance data to the control device via online connection or via manually assisted input when data is received via conventional means, e.g. paper and/or mail.

In combination with or as an alternative to the method and the system for controlling an individualized video data output on a display device, a system providing automated interventions may be provided.

In the method, the step of the receiving present individualized performance data may comprise receiving measured present individual performance data measured by a measuring or analyzing device. For example, a measuring device for measuring one or more of the following characteristics, specifically physiological and/or physical characteristics, may be used: body temperature, blood glucose value, blood pressure, vital lung capacity, body weight, and/or heart rate. Alternatively or in addition, a person's individual performance data used in the determination of the overall integrated patient performance may also include individual person performance data like, for example, daily activity rate, actual geographical position data, and/or health care related activities, like, for example, frequency of blood glucose monitoring, adherence to diagnostic procedures, adherence to medication, adherence to therapy plans, compliance with certain behavioral activities like for example: adherence to health screening schedules, adherence to diet plans, adherence to physical and mental activity plans, adherence to plans and/or schedules for social activities.

The display device may be provided in a device selected from the following group: tablet computer, watch, measuring wearable device, handheld display device, handheld measuring device, laptop computer, and personal computer.

Method according to one of the preceding claims, wherein the present individual performance data are representing present data for a group of characteristics selected from the following group of characteristics: biometric parameter, physiological parameter, geographical position data, physical activity and/or movement data, actually pursued check-up tests and/or screening procedures vs. planned check-up tests and/or screening procedures, A1c level value, Cholesterol value, blood sugar value, frequency of glucose monitoring vs. prescribed glucose monitoring, frequency of diagnostic monitoring vs. prescribed diagnostic monitoring, frequency and level of medication vs. prescribed frequency and level of medication, monitoring actual fitness activities vs. planned fitness activities, actual diet vs. planned diet, and actual social activities vs. planned social activities.

In the control device the different performance data may be weighted in classes.

Within the selected group of characteristics, the characteristics may be weighted equally or differently. Such weighting may be implemented by assigning weighting factors to at least one of the present performance data and the present performance parameter.

In an embodiment, the group of different video data output representations may comprise at least one video data output representation selected from the following group: icon, image, pattern of stripes, and pattern of circles. Video data output representations may refer to one and the same plurality of symbols or icons, but with different color representation.

Further, the method and/or the system may be used to provide frequent status updates to the individual based on the actual performance data measured as input from the input and/or measuring device. The frequent status update allows the individual to adapt the behavior based on the then current individual performance data represented by the Video data representations or by the representation of Video data of integrated values of performance data of sets of performance parameters. The set of used performance parameters used for the current status update to the individual may be changed over time and thus allows an adaptive and dynamically changing support mechanism to allow better self-management control.

The method and/or the system may be used to monitor performance data over a long period of time and thus provide an alert mechanism to the individual once performance data or sets of performance data of performance parameters lie outside a predetermined range of performance data values of one or a set of performance parameters. This alert mechanism may be used to trigger certain behaviors of an individual.

In an alternative, the method and/or the system may be used to monitor performance data over periods of time and thus provide a recording mechanism of health related behaviors as represented by the timely change of performance data or sets of performance data of a selected set of performance parameters. These recordings may be used to better understand the timely development of performance data or integrated values of sets of performance data and allow an understanding of the individual's underlying behaviors and thus allows providing health related advice and guidelines for the individual or groups of individuals.

Further, the method and/or the system may be used to monitor performance data of many individuals over periods of time in parallel and thus provide means to compute behavior patterns of groups of individuals. The patterns can be used to analyze and better understand the behavior of groups and compute group specific intervention plans and/or derive the effectiveness of interventions in correlation to the measured or inputted performance data. These analyses may be used to improve disease management or population health methods.

### DESCRIPTION OF PREFERRED EMBODIMENTS

Following, further embodiments will be described in further detail, by way of example, with reference to figures. In the figures show:

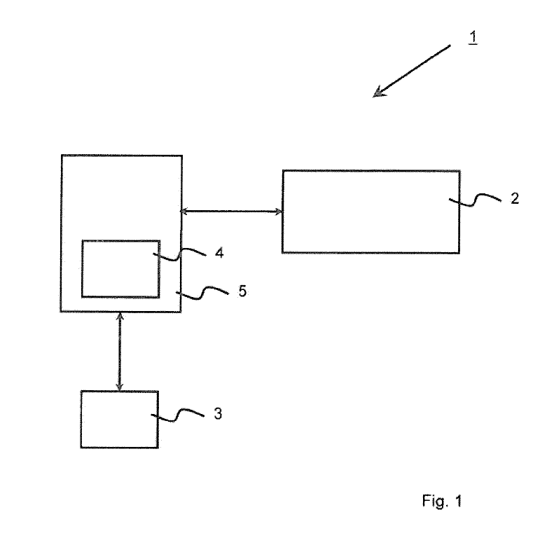

FIG. 1 a schematic representation of a system comprising a display device, an input device configured to receive present individual performance data, and a control device functionally connected to the input device and the display device,

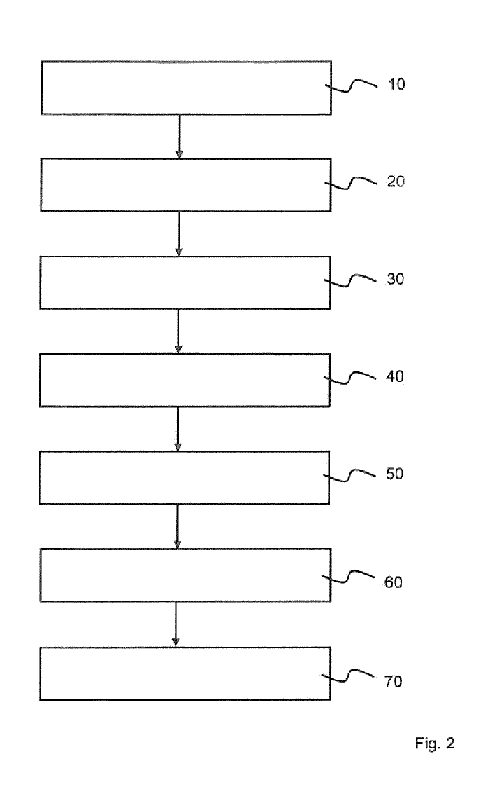

FIG. 2 a schematic representation of a method for controlling an individualized video data output on a display device of a system,

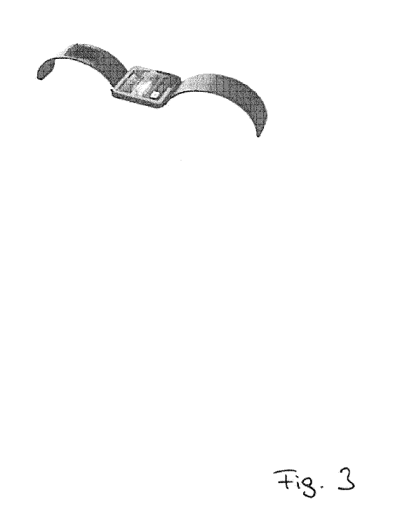

FIG. 3 a schematic representation of a watch having an electronic display for outputting individualized video data output representations,

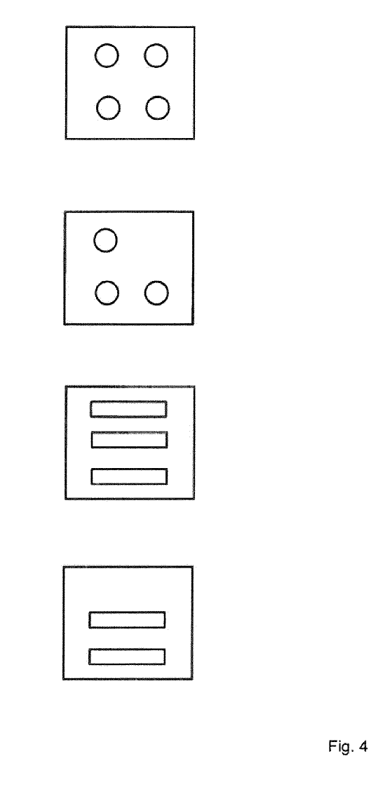

FIG. 4 a schematic representation of a plurality of video data output representations,

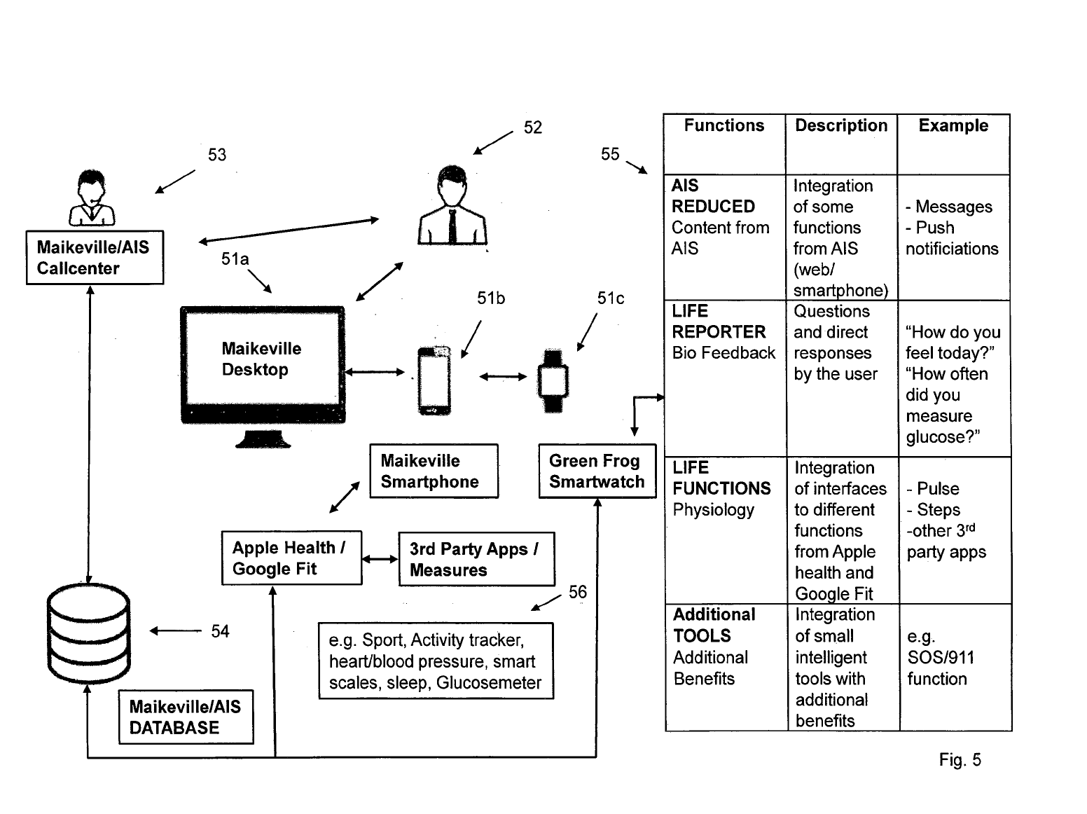

FIG. 5 a schematic representation of a further embodiment of a system implementation,

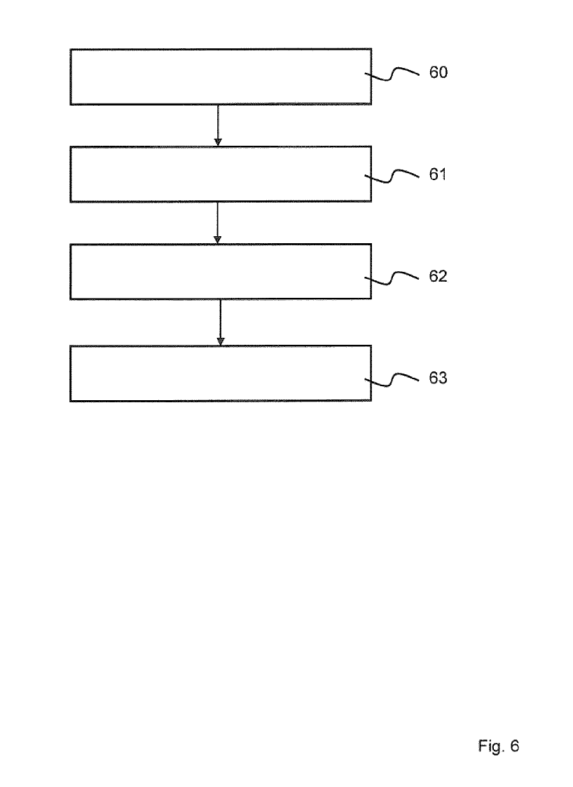

FIG. 6 a schematic representation of an overall process flow, and

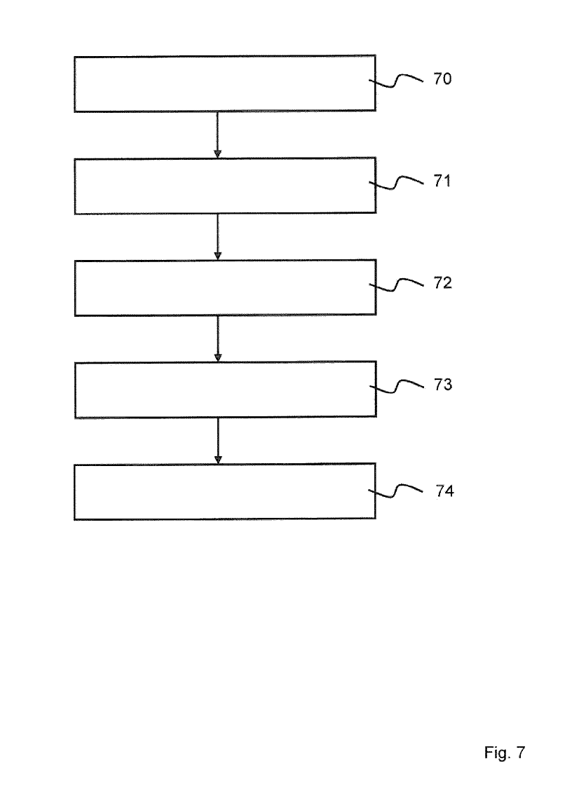

FIG. 7 a schematic representation of a process flow applied to messages sent to a user.

FIG. 1 shows a schematic representation of a system 1 comprising a display device 2, an input device 3 configured to receive present individual performance data, and a control device 4 functionally connected to the input device 3 and the display device 2. In the embodiment shown, the system 1 is implemented with a client-server architecture in which the control device 4 is provided in a central server device 5. The control device 4 comprises a data processing unit and a storage unit for storing electronic data. Data communication between the control device 4 and the display device 2 is done by wireless or wired based communication. The display device 2 may be provided in a device selected from the following group: tablet computer, watch (see FIG. 3), handheld display device, measuring wearable device, handheld measuring device, laptop computer, and personal computer.

The input device 3, for example, may be any touch sensitive device configured for user data input. As an alternative or in addition, the input device 3 may comprise any interface configured for receiving electronic data via wired and/or wireless data communication.

As an alternative, the control device 4 may be provided in a system component comprising the display 2 as well. For example, the display device 2, the input device 3, and the control device 4 may be provided in some common device housing. The watch shown in FIG. 3 may be suitable for receiving all or a subset of the functional devices in a single housing.

FIG. 2 shows a schematic representation of a method for controlling an individualized video data output on the display device 2 which may be performed by the system 1.

In the course of the method, in step 10 individualized or personalized video data output information is provided in the control device 4. The individualized video data output information comprises electronic data referring to a group of different video data output representations, each of the video data output representations representing a different video output on a display, e.g. an icon or a picture.

Further, the control unit is assigning values for integrated levels of performance data computed from values available for individualized groups of performance characteristics to the group of different video data output representations. The integration of selected sets of performance data is based on certain individualized algorithms resulting in one or more indicative integrated performance levels and/or performance categories. Thereby, an assigned video data output representation selected from the group of different video data output representations is determined for each of the resulting integrated performance levels or performance categories.

Referring to FIG. 2, in step 20 present individualized or individual performance data are received in the control device 4 via the input device 2. The present individualized performance data are indicative of a present performance of an individual, e.g. a patient. The performance of the individual, for example, may refer to physical, physiological, and/or psychological conditions of the individual. In general, a group of characteristics may be selected for determining the level of performance of the individual. For the characteristics selected present individual performance data are gathered, e.g. by measurements. The present individual performance data indicate the current individual's performance, e.g. a physical and/or psychological performance.

Following, in step 30 present individualized or individual performance parameters are determined by processing the present personalized performance data in the control device 4. The present individual performance parameters are generated for using such parameters.

The present personalized performance parameters may be derived from present data collected for at least one characteristic selected from the following group of characteristics: blood laboratory data, biometric parameter, physiological parameter, geographical position data, physical activity and/or movement data, actually pursued check-up tests and/or screening procedures maybe vs. planned check-up tests and/or screening procedures, A1c level value, Cholesterol value, blood sugar value, frequency of glucose monitoring maybe vs. prescribed glucose monitoring, frequency of diagnostic monitoring maybe vs. prescribed diagnostic monitoring, frequency and level of medication maybe vs. prescribed frequency and level of medication, monitoring actual fitness activities maybe vs. planned fitness activities, actual diet maybe vs. planned diet, and actual social activities maybe vs. planned social activities.

The personalized performance parameters derived are compared to the sets of performance parameters for determining a present video data output representation from the group of different video data output representations (step 40). The sets of performance parameters provided for and assigned to the group of different video data output representations in the control device may be provided in a memory device. As an alternative, an integrated performance level and/or category may be compared to the sets of performance levels and/or categories for determining a present video data output representation from the group of different video data output representations.

In step 50 video control signals are generated in the control unit 4. The video control signals generation may be done in response to the determination of the present video data output representation. The video control signals are configured to cause output of the present video data output representation on the display device 2.

In step 60 the video control signals are submitted to the display device 2.

In step 70 the present video data output representation is outputted on the display device 2. The present video data output representation can be represented in many ways. It can be as simple as showing a simple icon or graphic, or a changing background color or represented as a set of bars or as a number of bubbles (see FIG. 4) overlaid to the normal user interface of the display device (e.g. watch (see FIG. 3) or display of the portable device). The number of the bars or bubbles or their color may change reflecting the actual performance level of the user. In conclusion, the video data actually depicted on the display device 2 are selected in dependence on the performance parameter and/or the integrated performance level determined for the individual, i.e. the user or patient using the display device 2.

According to the technologies proposed here, a psychological system, which may also be referred to as Feeling (F) system may be used to stimulate specific brain functions and assist the individual to an improved disease management. For such stimulation the individualized video data output representations are used. With reducing the negative affect, which is caused by a depressing situation or general perception of the chronic disease, the utilization of the ‘Extension Memory’ can be improved through the process of the so called bio-feedback mechanism and thus turn the person towards a more problem solving behavior pattern.

When “turning on” the so-called F-System, people can be tuned to focus on “meaning” and “sense”. Using this stimulation, their expectation in using a supporting device (e.g. smart watch or smart phone) can be reflected and the function of the display device 2 can be used in an entirely novel way. A personalized icon can be established, which reflects the actual performance level. The control system has the ability to reflect the integral actual status by presenting a corresponding video data representation, which of course can change (e.g. color, shape, appearance) according to a potentially changing overall performance level.

For example the icon color may change from a preferred color when all is in check, to a personally less desirable color when things turn downhill or the display representation of the ‘positive behavior indicating icon’ disappears altogether to stimulate an activity of the user to get the desired symbol back by performing more appropriate health behavior. The icon can be a circle, a set of bars or number of points or even other representations reflecting a desired or comforting shape of the person.

The actual icon appearance is driven by the computed integrated performance level which threshold levels are set individually for a person (see below) and the selected set of performance criteria, which are used to compute the integrated performance level, can change over time as the general situation or behavior progress or decline of the person warrants.

The novel use of the system is demonstrated through its integrative approach of considering a broad variety of performance data to determine the actual integrative performance level. The user can look at the icon on his/her display device and is immediately informed about his/her individual performance level with an easy to grasp image representation. The novelty of the invention is also demonstrated in the flexible number and kind of considered performance parameters. The video output can have a changing meaning depending on what performance [parameters are currently important to improve the person's self-management or general health management.

The presented video output is dynamically updated on the display device 2 from the control device to reflect the user's actual performance. Looking at the icon the user is provided input for their ‘harmony seeking brain function’. Any deviation from the optimal (or normally expected picture) drives performance and motivation for improvements to resolve the conflict of expected picture and actual status. In essence, the method provides the user with a frequent realization of sense and meaning through the ‘bio-feedback’ effect of the icon's actual and expected appearance. The user's subconscious feelings can be expressed like: “My day getsa sense”, which is equivalent to make sure to get a representation of the expected and/or desired picture.

The icon changes and threshold levels for certain performance levels can be adapted remotely and dynamically through computation of the users present, past, and desired future performance characteristics and use of the system's control unit. This can be used for the users need and actual life situations. At times it might be easier to accomplish certain tasks and at other times it may be harder and necessary steps should be smaller, and therefore the control device can select optimal threshold values for performance levels and also select optimal video output representations.

The system provides a direct method for reducing health care cost by improving self-management, increasing compliance and enhancing overall engagement. It can potentially help individuals in slowing down disease progression, and improving their overall condition and happiness, and most importantly reduce inappropriate utilization and unnecessary hospitalization.

In an embodiment, the input device 3 by which the present individualized performance data are received may be any device configured for user data input like tablet computer, watch, handheld display device, laptop computer, and personal computer. The display device 2 can be a watch or a wristband with an integrated display device (see FIG. 3). The video data output representation may occur e.g. as a simple circle or five strips on the display of the watch or the wristband. The number of indicated stripes depends on the comparison between a desired and an actual value in the control device 4. The actual value depends on the present individualized performance data entered by the user via the input device 3. The present individualized performance data may comprise at least one individualized performance data selected from the following group: blood laboratory data, biometric parameter, physiological parameter, geographical position data, physical activity and/or movement data, actually pursued check-up tests and/or screening procedures maybe vs. planned check-up tests and/or screening procedures, A1c level value, Cholesterol value, blood sugar value, frequency of glucose monitoring maybe vs. prescribed glucose monitoring, frequency of diagnostic monitoring maybe vs. prescribed diagnostic monitoring, frequency and level of medication maybe vs. prescribed frequency and level of medication, monitoring actual fitness activities maybe vs. planned fitness activities, actual diet maybe vs. planned diet, and actual social activities maybe vs. planned social activities.

The control device 4 will access and value the individualized or individual performance data and with that determine the actual integrated performance levels. Some desired values of performance data (e.g. frequency of BG measurement; BG—blood glucose concentration) can be set by the user itself and will be stored in a storage device of the control device 4 until the user will change it.

A software module implemented on the control device 4 and providing an algorithm will determine desired values for sets of performance data. For it the individualized performance parameters may be divided in different classes. Biometric and physiological parameters may form a class. Frequency of glucose monitoring may form a class. Actual fitness activities may form a class. Actual diet as well as actual social activities may form a class. The class “fitness activities” may comprise performance data for e.g. swimming, running, walking. The class “diet” may comprise performance data for e.g. eating an apple, eating a salad. The class “social activities” may comprise performance data for e.g. meeting a friend, visits to the cinema, voluntary work.

The algorithm provided by the software module in the control device 4 may weigh the different performance data in the classes. In an example, swimming may be of higher importance than running and will get a relative higher weight than running. Running might be better than walking and will get a higher value than walking. The weighted personal performance parameter may be linked together in algorithms based on logical and/or rule based operations. A maximum desired value in a class can be formed e.g. by the value for swimming OR the value for walking AND running. Thus the maximum desired value of a class must not be the addition of every desired value of performance date of a class. The classes can be linked together as well. Thus the maximum desired value could e.g. be formed by the values for swimming OR running OR walking AND eating an Apple OR a salad AND measuring BG regular. The algorithm and the rule base can be updated and tuned according to the users progress or decline.

The control device 4 can have different opportunities to compare the actual with the desired values. The control device 4 can compare values directly: e.g. reported frequency of measurement BG with desired frequency of measuring, on the level of classes (e.g. reported fitness activity with desired fitness activity), or it can compare the sum of all reported values with the sum of all desired values. The result of the comparison of desired and reported data will determine the integrated performance level and the corresponding individualized video data output representation. For example, in one embodiment of the invention the performance level can be ranked: the closer the reported value comes to the desired value the higher will be the number of stripes on the display. The process is dynamic. If the user is falling behind with its activities and the value of reported data decreases in comparison to the desired value, the number of strips on the display will also decrease.

In another embodiment, the integrated performance level can be held and presented in a binary format: reaching a certain computed threshold integrated performance level results in one particular video representation and not reaching this level results in no or a different video representation.

The system and method described above may be implemented in an automated system for supporting people or to be more accurate patients with chronic diseases, to stay healthy. Such system may also be referred to as “Automated Intervention System” (AIS). AIS is an automated system, that enables an individual intervention performed automatically over an extended period of time with an initial and periodically repeated determination of the person's disposition along an eventually varying set of defined performance criteria. Because the AIS is automated and not requiring any substantial amount of manual adjustments it provides a low cost intervention model that helps individuals with chronic conditions improve their wellbeing through better self-management.

The widespread emergence of multiple communication channels provides a flexible means for reaching individuals. Personalization plays a central role in reducing the intrusiveness of how these new communication models are used to change behavior. The approach to personalization is based on psychographic profiling to better understand the individual and embedded knowledge to adapt an interaction to the individual's specific needs and capabilities.

For this purpose different testing procedures and proprietary surveys embedded in the AIS help to understand the patient concerning his personality traits, emotional state, medical conditions etc. The use of such testing procedures allows for determining the present individual performance data and determining the integrated performance level(s) in the control device 5 and retrieve the appropriate video output displayed in the display device 2.

Psychographic profiling provides insights into an individual's preferred interaction style as well as underlying behavioral inclinations. The system uses these insights to tailor the interaction to the individual. This same assessment and survey methodology reveals the underlying barriers that keep an individual from better self-management. The psychographic profiling and related survey technologies are used to design the intervention plan that is deployed for an individual.

FIG. 5 shows a schematic representation of a further embodiment of a system implementation 50. Aspects of the system 50 may be material for the realization of various embodiments, taken in isolation or in various combinations thereof.

The system 50 comprises user devices 51a, 51b, 51c provided as desktop computer, a tablet computer, and a mobile phone, respectively. The user devices 51a, 51b, 51c each are provided with a display device and a input device. The user 52 may interact with a call center 53, in addition to using one or more of the user device 51a, 51b, 51c. The user device 51a, 51b, 51c and the call center 53 are functionally connected to a server device 54 which also may provide for a data base by wired and/or wireless connection for data exchange.

A software module 55 (“GREEN FROGG”) is provided. Components of the software module may be implemented in the server device 54 and/or in the user device 51a, 51b, 51c. Third party applications 56 may interact with the server device 54 and/or the software module 55.

The system 50 may implement features of the embodiment depicted in FIG. 1 in part or as whole.

Messages play a central role in how the system is interacting with members. Messages sent to members may contain educational content, provide motivational support, establish trust between members and the AIS, and/or request the completion of educational and data entry tasks. The success of the system interventions may depend on how these messages are expressed. For this reason a distinction may be made between the topic of a message and the actual content used to express that topic.

The mapping between message topic and message content may be modulated based on the following message properties: Modality—Communication technology used to convey the message; Tonality—Intensity and tone used in the language that express the message; Timing—Frequency and number of messages and time interval between new messages and messages that have failed to generate a response. Once these properties are established, message content may be selected based on these properties plus the recipient's personality type and emotional state.

The individual message properties are determined by a method using an algorithm residing in the server unit 54. The method takes all measured performance data of the patient into account and calculates the set of message properties (modality, tonality, frequency) for each individual message. One key input of the computation for the message properties is provided by the result of one or more psychographic surveys, which the person has conducted. These surveys determine quantitatively psychological properties of the person on several psychological dimensions (e.g.: emotional, directional, analytical, contextual).

Following, a process flow which may be implemented by the system 50 is described.

Interactions with members by the initial AIS can be divided into phases: (i) Registration and Assessment Phase; (ii) Barrier Intervention Phase, and (iii) Post Intervention or Surveillance Phase.

The process flow may start with the registration and assessment processes, followed by the processes that address motivational or knowledge barriers detected during the assessment, and then shift to post intervention or surveillance processes. The post intervention process may continue interactions with the members, specifically patients, in order to maintain their engagement. This includes the collection of surveillance data that monitors the members' ongoing testing behavior. The post intervention interactions may continue until the person stops their participation. During the intervention phase and the post intervention phase the system 64 (FIG. 5) will continue to send personalized video data output information as provided in the control device 4 and displayed via the display device 2 (FIG. 1). The exception may be when a variance detected in the surveillance data indicates the emergence of a barrier that can be addressed by an intervention. Then the member is positioned back into the intervention phase. The member may also be referred to as user or patient.

FIG. 6 shows a schematic representation of an embodiment of an overall process flow. Aspects of the process flow may be material for the realization of various embodiments, taken in isolation or in various combinations thereof.

In the beginning of the process in step 60 the Registration and Assessment Phase is conducted. It begins by documenting the member's consent to participate in the project. The initial phase may continue by collecting a range of information that is used during the subsequent phases.

In step 61 additional assessment data are collected, and individual and or current barriers surveyed and detected.

In step 62 the initial Intervention Phase is pursued and continued as long as the system determines that a continuation is helpful for the person. This step 62 may conclude once the identified barriers have been appropriately addressed.

In step 63 the post-intervention or surveillance phase may continue to engage the member through the following interactions: Situation Specific Tips—Tips specific to the member's medical, social, or emotional situation; Motivational Messages—Messages designed to motivate the member based on his or her psychographic profile; Surveillance Data Collection—Member reported data characterizing compliance with the member's prescribed monitoring protocol.

Messages sent to the member may use a process flow for which an embodiment is depicted in FIG. 7. Aspects of the process flow may be material for the realization of various embodiments, taken in isolation or in various combinations thereof.

In step 70 the system determines tonality, modality and frequency as key qualifying and design parameters of the message. By doing this the message is tailored to member's psychographic profile and encourages the member's response when required by the message. The step that tailors the message properties uses logic that is encoded in a collection of if-then expressions residing in the software in the control device 5 (FIG. 1).

In step 71 the message content is composed from a collection of messages and/or message components prepared and stored in the data storage of the control device 5 (FIG. 1).

In step 72 the message is sent to the user along the modality determined in step 70 of the process.

In step 73 the system pauses according to the message frequency requirements as determined by the control device.

In step 74 the system determines if the objective of the intervention has been accomplished or not, based on the then actual and sometimes newly and repeatably measured performance data. If the person has not yet reached the desired performance and the system has determined that a continuation of pursuing the improvement process is intended then the process loops back to step 70 and starts over. Once the system has determined that a pursuit of the actual performance improvement is no longer necessary or intended by the systems rule set determined by the software residing in the control device 5 (FIG. 1) this particular barrier improvement process is considered closed and the system moves on to the next barrier or transitions into the surveillance phase (step 63, FIG. 6).

The System proposed may support several user groups: (1) Members, (2) Call Center Staff, and/or (3) System Administrators.

Following, further aspects of requirements for functionality modules of the system are described.

A member's primary interaction with the system may be through a web-based member application. This application will provide access to the member's Protected Healthcare Information (PHI). As such, access to the member application may be controlled by a two-factor authentication. The member application may be the program's primary face to the member. In a typical case, it is assumed that members will access the member application through internet browsers available on both large screen (e.g., computers and tablets) as well as small screen devices (e.g., Internet enabled smart phones).

That functionality for the members provided by the system may include: read/send message; join discussion forums; search educational content; review frequently asked questions; edit personal information; and/or help.

The functionalities for a call center may be: member search, and/or encounter summary (documentation & review of call center contacts with member).

So-called life reporter-features (Bio feedback) may be provided by the system, which is described above in the context of the selection of appropriate video output data representations.

With such features the system will receive information from the user about his emotional state and condition by different questions asked to the member like “How do you feel today?” The answers to the questions from system are provided by the member in an enjoyable and comfortable way using predominantly computer computable responses: for example a graphical slider on the input device, and/or by moving objects, buttons etc. Also the integration of a voice-out and voice-in functionality may be provided by the display device and the input device, respectively. Here the user can answer questions with his/her voice but also get messages via standard voice from the device. This functionality will help the users to get used to the system and to provide nearness.

So-called life functions (physiology) may be provided through the input device to allow the system to acquire additional performance data from the member. The additional performance data may include data like pulse, movement, and actual steps per time unit.

In addition the system may integrate data from third party applications to complement the data set of the person and thereby complementing the persons' actual or general performance data. Some examples of third party applications are: sport/activity tracker; heart/blood pressure products; smart scales; sleep tracker; and glucometer. All this data from third party applications already have an interface, so the person's data can be transferred to the AIS system via an API (application programmable interface).

The features disclosed in this specification, the figures and/or the claims may be material for the realization various embodiments, taken in isolation or in various combinations thereof.

1. A method for controlling an individualized video data output on a display device of a system further comprising an input device configured to receive present individual performance data and a control device functionally connected to the input device and the display device, the method comprising:
providing individualized video data output information in the control device, the individualized video data output information comprising a group of different video data output presentations and assigning a group of individual sets of performance parameters to the group of the different video data output presentations, thereby, for each of the individual sets of performance parameters defining an assigned video data output presentation selected from the group of different video data output presentations,
receiving present individual performance data in the control device via the input device, the present individual performance data being indicative of a present performance of an individual,
determining present individual performance parameters by processing the present individual performance data by the control device,
determining a present video data output presentation from the group of different video data output presentations by comparing the present individual performance parameters to the individual sets of performance parameters, and
generating video control signals in the control unit, the video control signals being configured to cause output of the present video data output representation on the display device, and
presenting the present video data output representation on the display device.

2. Method according to claim 1, wherein the system is implemented in a client-server system, the control device being provided in a central server device.

3. Method according to claim 1, wherein the receiving comprises receiving measured present individual performance data measured by a measuring device.

4. Method according to claim 1, wherein the display device is provided in a device selected from the following group: tablet computer, watch, handheld display device, handheld measuring device, laptop computer, and personal computer.

5. Method according to claim 1, wherein the present individual performance data are representing present data for a group of characteristics selected from the following group of characteristics: biometric parameter, physiological parameter, actually pursued check-up tests vs. planned check-up tests, A1c level value, blood sugar value, frequency of glucose monitoring vs. prescribed glucose monitoring, actual fitness activities vs. planned fitness activities, actual diet vs. planned diet, and actual social activities vs. planned social activities.

6. Method according to claim 1, wherein the group of different video data output presentations comprises at least one video data output presentation selected from the following group: icon, image, pattern of stripes, and pattern of circles.

7. A system for controlling an individualized video data output on a display device, the system comprising a display device, an input device configured to receive present individualized performance data, and a control device functionally connected to the input device and the display device, the system being configured to perform a method comprising:
providing individualized video data output information in the control device, the individualized video data output information comprising a group of different video data output presentations and assigning a group of individual sets of performance parameters to the group of the different video data output presentations, thereby, for each of the individual sets of performance parameters defining an assigned video data output presentation selected from the group of different video data output presentations,
receiving present individual performance data in the control device via the input device, the present individual performance data being indicative of a present performance of an individual,
determining present individual performance parameters by processing the present individual performance data by the control device,
determining a present video data output presentation from the group of different video data output presentations by comparing the present individual performance parameters to the individual sets of performance parameters, and
generating video control signals in the control unit, the video control signals being configured to cause output of the present video data output representation on the display device, and
presenting the present video data output representation on the display device.

8. A computer program product, stored on a storage medium and configured to perform the method according to claim 1 during operation on a data processing system.
